(19) United States
(12) Patent Application Publication
(10) Pub. No.: US 2021/0393624 A1
(43) Pub. Date: Dec. 23, 2021

(54) PROCESS FOR PRODUCING PHARMACEUTICAL DOSAGE FORMS CONTAINING TASK-1 AND TASK-3 CHANNEL INHIBITORS, AND THE USE OF SAME IN BREATHING DISORDER THERAPY

(71) Applicants: Bayer Aktiengesellschaft, Leverkusen (DE); Bayer Pharma Aktiengesellschaft, Berlin (DE)

(72) Inventors: Michelle STEIN, Wuppertal (DE); Moritz BECK-BROICHSITTER, Darmstadt (DE); Andrea ARNTZ, Düsseldorf (DE); Janine NICOLAI, Essen (DE)

(73) Assignees: Bayer Aktiengesellschaft, Leverkusen (DE); Bayer Pharma Aktiengesellschaft, Berlin (DE)

(21) Appl. No.: 17/296,914

(22) PCT Filed: Nov. 20, 2019
(86) PCT No.: PCT/EP2019/081950

§ 371 Date: May 25, 2021

(30) Foreign Application Priority Data
Nov. 27, 2018 (EP) 18208601.7

(51) Int. Cl. A61K 31/4995; A61K 47/26; A61K 47/10; A61K 31/496; A61K 9/00

(52) U.S. Cl. CPC A61K 31/4995; A61K 47/26; A61K 9/0043; A61K 31/496; A61K 47/10

(57) ABSTRACT

The invention relates to a process for producing pharmaceutical dosage forms containing potent and selective TASK-1 and/or TASK-3 channel inhibitors, and to the use of the dosage forms obtained by the said production process to treat and/or prevent breathing disorders, including sleep-related breathing disorders such as obstructive and central sleep apnea and snoring.

The present application relates to a process for producing pharmaceutical dosage forms comprising potent and selective inhibitors of TASK-1 and/or TASK-3 channels and to the use of the dosage forms obtained by the production process for the treatment and/or prevention of respiratory disorders, including sleep-related respiratory disorders such as obstructive and central sleep apneas and snoring.

Potassium channels are virtually ubiquitous membrane proteins which are involved in a large number of different physiological processes. This also includes the regulation of the membrane potential and the electric excitability of neurons and muscle cells. Potassium channels are divided into three major groups which differ in the number of transmembrane domains (2, 4 or 6). The group of potassium channels where two pore-forming domains are flanked by four transmembrane domains is referred to as K2P channels (Two-pore domain K⁺). Functionally, the K2P channels mediate, substantially time- and voltage-independently, K⁺ background currents, and their contribution to the maintenance of the resting membrane potential is crucial. The family of the K2P channels includes 15 members which are divided into six subfamilies, based on similarities in sequence, structure and function: TWIK (tandem pore domain halothane inhibited K⁺ channel), TREK (TWIK-related K⁺ channel), TASK (TWIK-related acid-sensitive K⁺ channel), TALK (TWIK-related alkaline pH activated K⁺ channel), THIK (tandem pore domain halothane inhibited K⁺ channel) and TRESK (TWIK-related spinal cord K⁺ channel).

Of particular interest are TASK-1 (KCNK3 or K2P3.1) and TASK-3 (KCNK9 or K2P9.1) of the TASK (TWIK-related acid-sensitive K⁺ channel) subfamily. Functionally, these channels are characterized in that, during maintenance of voltage-independent kinetics, they have “leak” or “background” currents flowing through them, and they respond to numerous physiological and pathological influences by increasing or decreasing their activity. Characteristic of TASK channels is the sensitive reaction to a change in extracellular pH: the channels are inhibited at acidic pH and activated at alkaline pH.

TASK-1 and TASK-3 channels play a role in respiratory regulation. Both channels are expressed in the respiratory neurons of the respiratory center in the brain stem, inter alia in neurons which generate the respiratory rhythm (ventral respiratory group with pre-Bötzinger complex), and in the noradrenergic Locus caeruleus, and also in serotonergic neurons of the raphe nuclei. Owing to the pH dependency, here the TASK channels have the function of a sensor which translates changes in extracellular pH into corresponding cellular signals [Bayliss et al., Pflugers Arch. 467, 917-929 (2015)]. TASK-1 and TASK-3 are also expressed in the Glomus caroticum, a paraganglion, which measures the pH and the 02 and CO₂ content of the blood and transmits signals to the respiratory center in the brain stem to regulate respiration. It was shown that TASK-1 knock-out mice have a reduced ventilatory response (increase of respiratory rate and tidal volume) to hypoxia and normoxic hypercapnia [Trapp et al., J. Neurosci. 28, 8844-8850 (2008)]. Furthermore, TASK-1 and TASK-3 channels were demonstrated in motoneurons of the Nervus hypoglossus, the XIIth cranial nerve, which has an important role in keeping the upper airways open [Berg et al., J. Neurosci. 24, 6693-6702 (2004)].

In a sleep apnea model in the anesthetized pig, nasal administration of a potassium channel blocker which blocks the TASK-1 channel in the nanomolar range led to inhibition of collapsibility of the pharyngeal airway musculature and sensitization of the negative pressure reflex of the upper airways. It is assumed that nasal administration of the potassium channel blocker depolarizes mechanoreceptors in the upper airways and, via activation of the negative pressure reflex, leads to increased activity of the musculature of the upper airways, thus stabilizing the upper airways and preventing collapse. By virtue of this stabilization of the upper airways, the TASK channel blockade may be of great importance for obstructive sleep apnea and also for snoring [Wirth et al., Sleep 36, 699-708 (2013); Kiper et al., Pflugers Arch. 467, 1081-1090 (2015)].

Obstructive sleep apnea (OSA) is a sleep-related respiratory disorder which is characterized by repeat episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper airways is substantially determined by the coordination and contraction property of the dilating muscles of the upper airways.

The Musculus genioglossus plays a decisive role in the pathogenesis of OSA. The activity of the Musculus genioglossus increases with decreasing pressure in the pharynx in the sense of a dilative compensation mechanism. Innervated by the Nervus hypoglossus, it drives the tongue forward and downward, thus widening the pharyngeal airway [Verse et al., Somnologie 3, 14-20 (1999)]. Tensioning of the dilating muscles of the upper airways is modulated inter alia via mechanoreceptors/stretch receptors in the nasal cavity/pharynx [Brouillette et al., J. Appl. Physiol. Respir. Environ. Exerc. Physiol. 49, 772-779 (1979)].

In sleeping patients suffering from serious sleep apnea, under local anesthesia of the upper airway an additional reduction of the activity of the Musculus genioglossus can be observed [Berry et al., Am. J. Respir. Crit. Care Med. 156, 127-132 (1997)]. Patients suffering from OSA have high mortality and morbidity as a result of cardiovascular disorders such as hypertension, myocardial infarction and stroke [Vrints et al., Acta Clin. Belg. 68, 169-178 (2013)].

In the case of central sleep apnea, owing to impaired brain function and impaired respiratory regulation there are episodic inhibitions of the respiratory drive. Central respiratory disorders result in mechanical respiratory arrests, i.e. during these episodes there is no breathing activity; temporarily, all respiratory muscles including the diaphragm are at rest. In the case of central sleep apnea, there is no obstruction of the upper airways.

In the case of primary snoring, there is likewise no obstruction of the upper airways. However, owing to the constriction of the upper airways, the flow rate of the air that is inhaled and exhaled increases. This, combined with the relaxed musculature, causes the soft tissues of the oral cavity and the pharynx to flutter in the stream of air. This gentle vibration then generates the typical snoring noises.

Obstructive snoring (upper airway resistance syndrome, heavy snoring, hypopnea syndrome) is caused by repeat partial obstruction of the upper airways during sleep. This results in an increased airway resistance and thus in an increase in work of breathing with considerable fluctuations in intrathoracic pressure. During inspiration, the development of negative intrathoracic pressure may reach values similar to those that are encountered as a result of complete airway obstruction during OSA. The pathophysiological consequences for heart, circulation and sleep quality correspond to those of obstructive sleep apnea. As in OSA, the pathogenesis can be assumed to be an impaired reflex mechanism of the pharynx-dilating muscles during inspiration when sleeping. Frequently, obstructive snoring is the preliminary stage of OSA [Hollandt et al., HNO 48, 628-634 (2000)].

The currently available therapeutic possibilities for snoring and OSA are limited. Mixtures of surface-active substances have been known since the 1980s which are intended to reduce the resistance of the upper airways and snoring [Widdicombe et al., Eur Resp J 1, 785-791 (1988)]. These mixtures comprise sodium chloride, glycerol, polysorbate 80 and benzalkonium chloride. From experiments in dogs, to which these mixtures were administered by injection into the pharynx, it was concluded that these mixtures reduce the resistance of the upper airways, increase the activity of the Musculus genioglossus when breathing in and breathing out and reduce snoring noises. OSA is not mentioned in the article by Widdicombe and it has also not been shown in this model that a collapse of the upper airways, which leads to apnea, could be prevented. The model of Widdicombe and Davies is therefore not predictive for OSA.

A composition based on glycerol, polysorbate 80, sodium chloride and 0.15% potassium sorbate (without benzalkonium chloride) is on the market as Asonor® as a therapy for snoring. In a study at University State Hospital in Copenhagen, the efficacy of nasal administration of Asonor® with respect to improving snoring was investigated in comparison with “Asonor®” without polysorbate 80. Both Asonor® and “Asonor®” without polysorbate 80 effected significant improvement of snoring [Report from the Department of Neurology, University State Hospital, Copenhagen, Denmark. The effect of nasal application of Asonor® and Polyglycoside 80 on snoring and sleep apnoea, 1989, [http://www.chrapat.sk/img/klinicka-dokumentacia.pdf.

WO-A 2012/010358 claims a pharmaceutical product comprising a container which comprises a liquid anti-snoring substance, wherein the container comprises a liquid outlet section which is configured to deliver the liquid anti-snoring substance directly into a nasal passage in the form of a jet stream. The liquid anti-snoring substance is an anti-snoring solution comprising sodium chloride, glycerol, polysorbate and sodium edetate and optionally potassium sorbate as preservative. WO-A 2012/010358 does not disclose a therapy of apnea or OSA. EP-B 2595685 describes the use of the described substance for the treatment of snoring and respiratory arrest (apnea).

No pharmacological therapy is currently available for therapy of OSA. Operations and oral devices are of only limited efficacy. The treatment standard is therapy with the continuous positive airway pressure (CPAP) system. However, the compliance rate of this therapy, due to the discomfort for the patient, is only 50-70% and the system is used on average not more than 4 hours per night.

Novel substances, which act as potent and selective inhibitors of TASK-1 and/or TASK-3 channels and are suitable as such in particular for the treatment and/or prevention of respiratory disorders, including sleep-related respiratory disorders such as obstructive and central sleep apneas and snoring and also other disorders, are known from WO-A 2017/097792 and WO-A 2017/097671, WO-A 2018/015196, EP 17176046.5 (unpublished) and PCT/CN2017/088237 (unpublished). The potent and selective inhibitors of TASK-1 and/or TASK-3 channels disclosed therein thus represent an alternative to the CPAP system for the treatment of sleep-related respiratory disorders such as obstructive and central sleep apneas and snoring. Thus, the potent and selective inhibitors of TASK-1 and/or TASK-3 channels can increase the rate of compliance by the patients of a treatment and/or prevention of respiratory disorders, including sleep-related respiratory disorders such as obstructive and central sleep apneas and snoring, compared to the current therapy standard (therapy of OSA: CPAP system). For this purpose, this alternative therapy should be simple and comfortable to use and not disturb the person sleeping. In addition, this alternative therapy should enable an undisturbed night's rest without repeat medication with a once daily dose prior to going to sleep.

The potent and selective inhibitors of TASK-1 and/or TASK-3 channels known from WO-A 2017/097792 and WO-A 2017/097671, WO-A 2018/015196, EP 17176046.5 (unpublished) and PCT/CN2017/088237 (unpublished) are light-, temperature- and oxidation-sensitive active compounds suffering unwanted degradation during the preparation of the formulation and during storage in aqueous unbuffered solution. In addition, the known potent and selective inhibitors of TASK-1 and/or TASK-3 channels have a poor solubility in water which is insufficient for accommodating the amount of active ingredient required for pharmacological activity in the limited administration volume (in the case of nasal administration about 50 to 300 μl). Using solubilizers approved and/or known for the nasal or pharyngeal administration route such as, for example, cosolvents (e.g. polyethylene glycol 400 (PEG400)) or surfactants (e.g. polysorbate 80), it is possible to achieve the desired solubilities; however, in spite of the presence of the known solubilizers, in aqueous solution a low dissolution rate of the known potent and selective inhibitors of the TASK-1 and/or TASK-3 channels is observed, which leads to significantly prolonged processing times during production and the associated unwanted degradation of the active compound used.

The aqueous formulations known from WO-A 2018/114501 and WO-A 2018/114503 of the potent and selective inhibitors of the TASK-1 and/or TASK-3 channels comprise polyethylene glycol 400 or glycerol, at least one auxiliary selected from the group of the pH regulators, at least one auxiliary selected from the group of the solubilizers, at least one auxiliary selected from the group of the stabilizers.

The formulations described in the prior art comprising inhibitors of the TASK-1 and/or TASK-3 channels have the disadvantage that during the production process degradation products of the active compound may already be formed, and unwanted discolorations may occur. Also, there may be an increased degradation of the active compound during storage. Furthermore, the production of the formulations, even in small amounts of about 100 ml, is associated with a considerable expenditure of time (>24 h) caused by the only insufficient solubility of the active compounds.

Furthermore, the pharmacologically active substances for the treatment and/or prevention of respiratory disorders should be present in a pharmaceutical formulation which is perceived as having a neutral taste, in particular also in a comparison between pharmaceutical formulations comprising the pharmacologically active substances (verum) and those not comprising any (placebo).

Accordingly, it was the object of the present invention to provide an improved process for the production of stable aqueous formulations based on potent and selective inhibitors of the TASK-1 and/or TASK-3 channels for the treatment and/or prevention of respiratory disorders, including sleep-related respiratory disorders such as obstructive and central sleep apneas and snoring, which ensures sufficient stability of the active compound in the resulting aqueous formulation during production and storage and additionally allows the active compound to be dissolved in sufficiently high concentrations without any undesirably high time expenditure during the production process.

In addition, it was another object of the present invention to provide aqueous formulations based on potent and selective inhibitors of the TASK-1 and/or TASK-3 channels for the treatment and/or prevention of respiratory disorders, including sleep-related respiratory disorders such as obstructive and central sleep apneas and snoring, which are perceived as having a neutral taste.

Surprisingly, it has been found that the dissolution rate of the potent and selective inhibitors of the TASK-1 and/or TASK-3 channels can be shortened and the time for producing aqueous formulations thereby reduced significantly when inhibitors of the TASK-1 and/or TASK-3 channels are pre-dissolved in the surfactant (e.g. polysorbate 80) and/or cosolvents (e.g. PEG 400). Here, it has likewise surprisingly been found that unwanted discolorations and degradation products during production and storage of the formulation can be avoided when the stabilizer is likewise pre-dissolved in the mixture of surfactant and/or cosolvents and only then the inhibitors of the TASK-1 and/or TASK-3 channels are added.

A further unexpected effect of the formulations according to the invention is that they can mask the taste component of the surfactants and the pharmacologically active substances used by employing sweeteners.

The present invention provides a process for the production of stable pharmaceutical formulations, characterized in that

in a first step at least one polyoxyethylenesorbitan fatty ester is initially charged as solubilizer and/or PEG 400 is initially charged as cosolvent, at least one antioxidant and a therapeutically effective amount of at least one inhibitor of the TASK-1 and/or TASK-3 channel or a hydrate, solvate, polymorph or metabolite thereof or a pharmaceutically acceptable salt are dissolved therein and subsequently at least one pH regulator, water and optionally glycerol, polyoxyethylene sorbitan fatty ester or PEG400 and optionally at least one sweetener are added, the pH of the resulting solution being between 6.8 and 8.2, preferably from 6.8 to 7.8.

The present invention provides pharmaceutical formulations obtainable by the process according to the invention.

In a preferred embodiment of the process according to the invention, at least one polyoxyethylene sorbitan fatty ester as solubilizer and/or PEG 400 as cosolvent is initially charged, the antioxidant is then added and subsequently a therapeutically effective amount of at least one inhibitor of the TASK-1 and/or TASK-3 channel or a hydrate, solvate, polymorph or metabolite thereof or a pharmaceutically acceptable salt is dissolved therein.

In a further preferred embodiment of the process according to the invention, initially
a primary solution (A) comprising at least one polyoxyethylene sorbitan fatty ester (polysorbate) and/or PEG 400 and also at least one antioxidant is prepared and, in a further step, a therapeutically effective amount of at least one inhibitor of the TASK-1 and/or TASK-3 channel or a hydrate, solvate, polymorph or metabolite thereof or a pharmaceutically acceptable salt is dissolved in this mixture, and this is added to a
solution (B) comprising at least one pH regulator, water and optionally glycerol or PEG400 and optionally at least one sweetener, the pH of the resulting solution being between 6.8 and 8.2, preferably from 6.8 to 7.8.

Preferably, the addition of the primary solution (A) to solution (B) takes place over a period of 15 to 30 min, preferably within 30 min. It is also possible to transfer solution (B) into the pre-solution (A).

In a particularly preferred embodiment of the process according to the invention, in a first step at least one polyoxyethylene sorbitan fatty ester is initially charged, the antioxidant is then added and subsequently a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone is dissolved therein and then at least one pH regulator, optionally at least one sweetener and water are added.

In a very particularly preferred embodiment of the process according to the invention, in a first step at least one polyoxyethylene sorbitan fatty ester is initially charged, the antioxidant is then added and subsequently a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone is dissolved therein and then at least one pH regulator, at least one sweetener and water are added.

In a likewise very particularly preferred embodiment of the process according to the invention, in a first step at least one polyoxyethylene sorbitan fatty ester is initially charged, the antioxidant is then added and subsequently a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone is dissolved therein and then at least one pH regulator and water are added.

In a further particularly preferred embodiment of the process according to the invention, in a first step at least one polyoxyethylene sorbitan fatty ester is initially charged, the antioxidant is then added and subsequently a therapeutically effective amount of (4-{[2-(4-chlorophenyl)imidazo[1,2-a]pyridin-3-yl]methyl}piperazin-1-yl)(6-methoxypyridin-2-yl)methanone is dissolved and then at least one pH regulator, glycerol or PEG 400, optionally a sweetener and water are added.

In a further particularly preferred embodiment of the process according to the invention, in a first step PEG 400 is initially charged, the antioxidant is then added and subsequently a therapeutically effective amount of (4-{[2-(4-chlorophenyl)imidazo[1,2-a]pyridin-3-yl]methyl}piperazin-1-yl)(6-methoxypyridin-2-yl)methanone is dissolved and then at least one pH regulator, at least one polyoxyethylene sorbitan fatty ester, optionally a sweetener and water are added.

In a further particularly preferred embodiment of the process according to the invention, in a first step at least one polyoxyethylene sorbitan fatty ester is initially charged, the antioxidant is then added and subsequently a therapeutically effective amount of (4-{[2-(4-chlorophenyl)imidazo[1,2-a]pyridin-3-yl]methyl}piperazin-1-yl)(6-methoxypyridin-2-yl)methanone is dissolved and then at least one pH regulator, glycerol or PEG 400, at least one sweetener and water are added.

The pharmaceutical formulations according to the invention comprise at least one polyoxyethylene sorbitan fatty ester, at least one antioxidant and a therapeutically effective amount of at least one inhibitor of the TASK-1 and/or TASK-3 channel or a hydrate, solvate, polymorph or metabolite thereof or a pharmaceutically acceptable salt, optionally glycerol or PEG 400 and optionally at least one sweetener, at least one pH regulator and water, the pH of the resulting solution being between 6.8 and 8.2, preferably from 6.8 to 7.8.

Preferably, the pharmaceutical formulations according to the invention comprise at least one polyoxyethylene sorbitan fatty ester (polysorbate) as solubilizer and/or a cosolvent, at least one antioxidant and a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, at least one sweetener, optionally at least one pH regulator and water, the pH of the resulting solution being between 6.8 and 8.2, preferably from 6.8 to 7.8.

Particularly preferably, the pharmaceutical formulations according to the invention comprise at least one polyoxyethylene sorbitan fatty ester (polysorbate) as solubilizer and/or a cosolvent, at least one antioxidant and a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, at least one sweetener, at least one pH regulator and water, the pH of the resulting solution being between 6.8 and 8.2, preferably from 6.8 to 7.8.

Particularly preferably, the pharmaceutical formulations according to the invention comprise at least one polyoxyethylene sorbitan fatty ester (polysorbate) as solubilizer and/or a cosolvent, at least one antioxidant and a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone, optionally a sweetener, at least one pH regulator and water, the pH of the resulting solution being between 6.8 and 8.2, preferably from 6.8 to 7.8.

In an embodiment (A), the formulation according to the invention comprises
1 to 21% by weight of a polyoxyethylene sorbitan fatty ester,
0.001 to 0.20% by weight of an antioxidant,
0.002 to 0.10% by weight of a therapeutically effective amount of at least one inhibitor of the TASK-1 and/or TASK-3 channel or a hydrate, solvate, polymorph or metabolite thereof or a pharmaceutically acceptable salt,
0.3 to 25% by weight of glycerol and
53.5 to 98% by weight of a buffer solution having a substance concentration of 25 to 200 mM.

Preferably, the formulation (A) according to the invention comprises
2.5 to 16% by weight of a polyoxyethylene sorbitan fatty ester,
0.002 to 0.1% by weight of an antioxidant,
0.005 to 0.07% by weight of a therapeutically effective amount of at least one inhibitor of the TASK-1 and/or TASK-3 channel or a hydrate, solvate, polymorph or metabolite thereof or a pharmaceutically acceptable salt,
1 to 12% by weight of glycerol and
71 to 96% by weight of a buffer solution having a substance concentration of 25 to 200 mM.

Very particularly preferably, the formulation (A) according to the invention comprises
5 to 13% by weight of a polyoxyethylene sorbitan fatty ester,
0.005 to 0.05% by weight of an antioxidant,
0.006 to 0.06% by weight of a therapeutically effective amount of at least one inhibitor of the TASK-1 and/or TASK-3 channel or a hydrate, solvate, polymorph or metabolite thereof or a pharmaceutically acceptable salt,
2 to 6% by weight of glycerol and
80.5 to 92% by weight of a buffer solution having a substance concentration of 25 to 200 mM.

In a further embodiment (B), the formulation according to the invention comprises
1 to 20% by weight of a polyoxyethylene sorbitan fatty ester,
0.001 to 0.2% by weight of an antioxidant,
0.002 to 0.10% by weight of a therapeutically effective amount of at least one inhibitor of the TASK-1 and/or TASK-3 channel or a hydrate, solvate, polymorph or metabolite thereof or a pharmaceutically acceptable salt,
3.0 to 60% by weight of PEG 400 and
19 to 95.5% by weight of a buffer solution having a substance concentration of 25 to 200 mM.

Preferably, the formulation (B) according to the invention comprises
2.5 to 15% by weight of a polyoxyethylene sorbitan fatty ester,
0.002 to 0.10% by weight of an antioxidant,
0.005 to 0.07% by weight of a therapeutically effective amount of at least one inhibitor of the TASK-1 and/or TASK-3 channel or a hydrate, solvate, polymorph or metabolite thereof or a pharmaceutically acceptable salt,
6 to 36% by weight of PEG 400 and
48 to 91% by weight of a buffer solution having a substance concentration of 25 to 200 mM.

Particularly preferably, the formulation (B) according to the invention comprises
to 13% by weight of a polyoxyethylene sorbitan fatty ester,
0.005 to 0.05% by weight of an antioxidant,
0.006 to 0.06% by weight of a therapeutically effective amount of at least one inhibitor of the TASK-1 and/or TASK-3 channel or a hydrate, solvate, polymorph or metabolite thereof or a pharmaceutically acceptable salt,
12 to 23.5% by weight of PEG 400 and
63 to 82.5% by weight of a buffer solution having a substance concentration of 25 to 200 mM.

Very particularly preferably, the formulations (A) and (B) according to the invention comprise, as inhibitor of the TASK-1 and/or TASK-3 channel, 4-{[2-(4-chlorophenyl)imidazo[1,2-a]pyridin-3-yl]methyl}piperazin-1-yl)(6-methoxypyridin-2-yl)methanone.

In a further embodiment (C), the formulation according to the invention comprises
1 to 21% by weight of a polyoxyethylene sorbitan fatty ester,
0.001 to 0.2% by weight of an antioxidant,
0.002 to 0.1% by weight of a therapeutically effective amount of at least one inhibitor of the TASK-1 and/or TASK-3 channel or a hydrate, solvate, polymorph or metabolite thereof or a pharmaceutically acceptable salt,
0.01 to 6% by weight of a sweetener and
72 to 98.5% by weight of a buffer solution having a substance concentration of 25 to 200 mM.

Preferably, the formulation (C) according to the invention comprises
2.5 to 16% by weight of a polyoxyethylene sorbitan fatty ester,
0.002 to 0.1% by weight of an antioxidant,
0.005 to 0.07% by weight of a therapeutically effective amount of at least one inhibitor of the TASK-1 and/or TASK-3 channel or a hydrate, solvate, polymorph or metabolite thereof or a pharmaceutically acceptable salt,
0.02 to 1% by weight of a sweetener and
82.5 to 97% by weight of a buffer solution having a substance concentration of 25 to 200 mM.

Particularly preferably, the formulation (C) according to the invention comprises
to 13% by weight of a polyoxyethylene sorbitan fatty ester,
0.005 to 0.05% by weight of an antioxidant,
0.006 to 0.06% by weight of a therapeutically effective amount of at least one inhibitor of the TASK-1 and/or TASK-3 channel or a hydrate, solvate, polymorph or metabolite thereof or a pharmaceutically acceptable salt,
0.05 to 0.2% by weight of a sweetener and
86 to 94% by weight of a buffer solution having a substance concentration of 25 to 200 mM.

Very particularly preferably, the formulation (C) according to the invention comprises, as inhibitor of the TASK-1 and/or TASK-3 channel, (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

In a further embodiment (C′), the formulation according to the invention comprises
1.4 to 22.7% by weight of a polyoxyethylene sorbitan fatty ester,
0.001 to 0.2% by weight of an antioxidant,
0.002 to 0.1% by weight of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone or a hydrate, solvate, polymorph or metabolite thereof or a pharmaceutically acceptable salt,
0 to 4% by weight of a sweetener and
73 to 98.5% by weight of a buffer solution having a substance concentration of 25 to 200 mM.

Preferably, the formulation (C′) according to the invention comprises
2.9 to 15.8% by weight of a polyoxyethylene sorbitan fatty ester,
0.002 to 0.1% by weight of an antioxidant,
0.005 to 0.07% by weight of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone or a hydrate, solvate, polymorph or metabolite thereof or a pharmaceutically acceptable salt,
0.01 to 2% by weight of a sweetener and
82 to 97% by weight of a buffer solution having a substance concentration of 25 to 200 mM.

Particularly preferably, the formulation (C′) according to the invention comprises
5.4 to 13% by weight of a polyoxyethylene sorbitan fatty ester,
0.005 to 0.05% by weight of an antioxidant,
0.006 to 0.05% by weight of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone or a hydrate, solvate, polymorph or metabolite thereof or a pharmaceutically acceptable salt,
0.02 to 1% by weight of a sweetener and
85.9 to 94.5% by weight of a buffer solution having a substance concentration of 25 to 200 mM.

In a further embodiment (D), the formulation according to the invention comprises
1 to 21% by weight of a polyoxyethylene sorbitan fatty ester,
0.001 to 0.2% by weight of an antioxidant,
0.002 to 0.1% by weight of a therapeutically effective amount of at least one inhibitor of the TASK-1 and/or TASK-3 channel or a hydrate, solvate, polymorph or metabolite thereof or a pharmaceutically acceptable salt,
0.3 to 25% by weight of glycerol
0.01 to 5.5% by weight of a sweetener and
48 to 98% by weight of a buffer solution having a substance concentration of 25 to 200 mM.

Preferably, the formulation (D) according to the invention comprises
2.5 to 16% by weight of a polyoxyethylene sorbitan fatty ester,
0.002 to 0.1% by weight of an antioxidant,
0.005 to 0.07% by weight of a therapeutically effective amount of at least one inhibitor of the TASK-1 and/or TASK-3 channel or a hydrate, solvate, polymorph or metabolite thereof or a pharmaceutically acceptable salt,
1 to 12% by weight of glycerol
0.02 to 1% by weight of a sweetener and
70 to 96% by weight of a buffer solution having a substance concentration of 25 to 200 mM.

Particularly preferably, the formulation (D) according to the invention comprises
to 13% by weight of a polyoxyethylene sorbitan fatty ester,
0.005 to 0.05% by weight of an antioxidant,
0.006 to 0.06% by weight of a therapeutically effective amount of at least one inhibitor of the TASK-1 and/or TASK-3 channel or a hydrate, solvate, polymorph or metabolite thereof or a pharmaceutically acceptable salt,
2.5 to 6% by weight of glycerol
0.05 to 0.3% by weight of a sweetener and
80 to 92% by weight of a buffer solution having a substance concentration of 25 to 200 mM.

Very particularly preferably, the formulation (D) according to the invention comprises, as inhibitor of the TASK-1 and/or TASK-3 channel, 4-{[2-(4-chlorophenyl)imidazo[1,2-a]pyridin-3-yl]methyl}piperazin-1-yl)(6-methoxypyridin-2-yl)methanone.

Suitable polyoxyethylene sorbitan fatty esters according to the invention are, for example, polysorbate 20, polysorbate 40, polysorbate 60, polysorbate 80. Preference is given to polysorbate 80.

Suitable antioxidants are, for example, butylated hydroxyanisole or butylated hydroxytoluene. Preference is given to butylated hydroxyanisole.

Suitable sweeteners according to the invention are, for example, sucralose or sorbitol. Preference is given to sucralose.

In a preferred embodiment, the antioxidant, preferably butylated hydroxyanisole, is present in comminuted form; with particular preference, the crystals have a diameter of less than 1 mm.

In a preferred embodiment, the inhibitor of the TASK-1 and/or TASK-3 channel or a hydrate, solvate, polymorph or metabolite thereof or a pharmaceutically acceptable salt is present in micronized form with a mean particle size (x₅₀) of from 1 to 8 μm and an upper limit (x₉₀) of 20 μm.

Preference is given to using a buffer solution selected from the group comprising phosphate buffer, 2-(4-(2-hydroxyethyl)-1-piperazinyl)ethanesulfonic acid (HEPES), 2-amino-2-(hydroxymethyl)propane-1,3-diol (TRIS) and 3-(N-morpholino)propanesulfonic acid (MOPS).

Particularly preferably, the phosphate buffer solution comprises sodium dihydrogen phosphate dihydrate and disodium hydrogen phosphate and water at a pH of 7.0.

Particularly preferably, the HEPES buffer solution comprises 2-(4-(2-hydroxyethyl)-1-piperazinyl)ethanesulfonic acid and water, adjusted to pH 7.6 with aqueous sodium hydroxide solution.

Particularly preferably, the TRIS buffer solution comprises 2-amino-2-(hydroxymethyl)propane-1,3-diol and water, adjusted to pH 8.0 with hydrochloric acid.

Particularly preferably, the MOPS buffer solution comprises 3-(N-morpholino)propanesulfonic acid and water, adjusted to pH 7.5 with aqueous sodium hydroxide solution.

Very particularly preferably, the pH regulator is a phosphate buffer solution or 2-(4-(2-hydroxyethyl)-1-piperazinyl)ethanesulfonic acid (HEPES).

The present invention provides pharmaceutical formulations for nasal or pharyngeal administration, obtainable by the process according to the invention.

The stable pharmaceutical formulations according to the invention may optionally comprise further auxiliaries.

Examples of auxiliaries in the context of the present invention are stabilizers, thickeners, preservatives, substances for adjusting tonicity, aromas, fragrances or dyes.

In the context of the present invention, thickeners are, for example, natural rubbers, alginic acid, pectins, starch and starch derivatives, gelatins, poloxamers (polyoxypropylene/polyoxyethylene block copolymers), cellulose derivatives, acrylic acid polymers or vinyl polymers.

In the context of the present invention, an active ingredient is defined as an inhibitor of the TASK-1 and/or TASK-3 channel, or a hydrate, solvate, polymorph, or metabolite thereof or a pharmaceutically acceptable salt thereof.

Stable pharmaceutical formulations according to the invention are, for example, those formulations in which the at least one inhibitor of the TASK-1 and/or TASK-3 channel is selected from the compounds of the formula (I) described in WO 2017/097671, WO 2017/097792, WO 2018/015196 and EP17176046.5 and PCT/CN2017/088237 and their salts, solvates and solvates of the salts. The synthesis of these compounds is described in WO 2017/097792.

Preferred compounds of the formula (I) are selected from the group comprising compounds of Table 1: Compounds from WO 2017/097671, WO 2017/097792, WO 2018/015196 and EP17176046.5 and PCT/CN2017/088237

| Example | Name |
|---|---|
| 1 | WO 2017/097671, Ex. 4: [5-{[2-(4-chlorophenyl)imidazo[1,2-a]pyridin-3- |
| | yl]methyl}hexahydropyrrolo[3,4-c]pyrrol-2(1H)-yl](6-methoxypyridin-2-yl)methanone |
| 2 | WO 2017/097671, Ex. 10: [[5-{[2-(4-bromophenyl)imidazo[1,2-a]pyridin-3 |
| | yl]methyl}hexahydropyrrolo[3,4-c]pyrrol-2(1H)-yl](3-methoxyphenyl)methanone |
| 3 | WO 2017/097671, Ex. 24: [5-{[2-(4-bromophenyl)imidazo[1,2-a]pyridin-3 |
| | yl]methyl}hexahydropyrrolo[3,4-c]pyrrol-2(1H)-yl](6-methoxypyridin-2-yl)methanone |
| 4 | WO 2017/097671, Ex. 26: (2-fluorophenyl)[5-{[2-(4-isopropylphenyl)-imidazo[1,2- |
| | a]pyridin-3-yl]methyl}hexahydropyrrolo[3,4-c]pyrrol-2(1H)-yl]methanone |
| 5 | WO 2017/097792, Ex. 2: (4-{[2-(4-chlorophenyl)imidazo[1,2- |
| | a]pyridin-3-yl]methyl}piperazin-1-yl)(cyclopentyl)methanone |
| 6 | WO 2017/097792, Ex. 3: (4-{[2-(4-chlorophenyl)imidazo[1,2- |
| | a]pyridin-3-yl]methyl}piperazin-1-yl)(6-methoxypyridin-2-yl)methanone |
| 7 | WO 2017/097792, Ex. 4: (4-{[2-(4-bromophenyl)imidazo[1,2- |
| | a]pyridin-3-yl]methyl}piperazin-1-yl)(2-fluorophenyl)methanone |
| 8 | WO 2017/097792, Ex. 42: (3-bromo-6-methoxypyridin-2-yl)(4-{[2-(4- |
| | chlorophenyl)imidazo[1,2-a]pyridin-3-yl]methyl}piperazin-1-yl)methanone |
| 9 | WO 2017/097792, Ex. 43: (3-chloro-6-methoxypyridin-2-yl)(4-{[2-(4- |
| | chlorophenyl)imidazo[1,2-a]pyridin-3-yl]methyl}piperazin-1-yl)methanone |
| 10 | WO 2018/015196, Ex. 21: (−)-[(1S,4S)-5-{[2-(4-chlorophenyl)imidazo[1,2-a]pyridin-3- |
| | yl]methyl}-2,5-diazabicyclo[2.2.2]oct-2-yl](6-methoxypyridin-2-yl)methanone |
| | (enantiomer 1) |
| 11 | WO 2018/015196, Ex. 24: (5-{[2-(5-chloropyridin-2-yl)imidazo[1,2-a]pyridin-3-yl]methyl}- |
| | 2,5-diazabicyclo[2.2.2]oct-2-yl)-(3-fluoro-6-methoxypyridin-2-yl)methanone (enantiomer 1) |
| 12 | WO 2018/015196, Ex. 26: (−)-(5-{[2-(5-chloropyridin-2-yl)imidazo[1,2-a]pyridin-3- |
| | yl]methyl}-2,5-diazabicyclo[2.2.2]oct-2 yl)-(6-methoxypyridin-2-yl)methanone |
| | (enantiomer 1) |
| 13 | WO 2018/015196, Ex. 29: (7-{[2-(4-chlorophenyl)imidazo[1,2-a]pyridin-3-yl]methyl}-3- |
| | oxa-7,9-diazabicyclo[3.3.1]non-9 yl)-(6-methoxypyridin-2-yl)methanone |
| 14 | WO 2018/015196, Ex. 126: (3-fluoro-6-methoxypyridin-2-yl)(5-{[2-(6-isopropylpyridin-3- |
| | yl)imidazo[1,2-a]pyridin-3-yl]methyl}-2,5-diazabicyclo[2.2.2]oct-2-yl)methanone |
| | (enantiomer 1) |
| 15 | WO 2018/015196, Ex. 200: (5-{[2-(5-chloropyridin-2-yl)imidazo[1,2-a]pyridin-3- |
| | yl]methyl}-2,5-diazabicyclo[2.2.2]oct-2-yl)-[6-(difluoromethoxy)pyridin-2-yl]methanone |
| | (enantiomer 1) |
| 16 | EP17176046.5, Ex. 24: (5-{[2-(4-chlorophenyl)imidazo[1,2-a]pyrimidin-3-yl]methyl}-2,5- |
| | diazabicyclo[2.2.2]oct-2-yl)-(3-fluoro-6-methoxypyridin-2-yl)methanone (enantiomer 2) |
| 17 | EP17176046.5, Ex. 26: (3-chloro-6-methoxypyridin-2-yl)(5-{[2-(4- |
| | isopropylphenyl)imidazo[1,2-a]pyrimidin-3-yl]methyl}-2,5-diazabicyclo[2.2.2]oct-2- |
| | yl)methanone (enantiomer 2) |
| 18 | EP17176046.5, Ex. 28: (3-fluoro-6-methoxypyridin-2-yl)(3-{[2-(4- |
| | isopropylphenyl)imidazo[1,2-a]pyrimidin-3-yl]methyl}-3,8-diazabicyclo[3.2.1]oct-8- |
| | yl)methanone |
| 19 | EP17176046.5, Ex. 29: (3-chloro-6-methoxypyridin-2-yl)(3-{[2-(4- |
| | isopropylphenyl)imidazo[1,2-a]pyrimidin-3-yl]methyl}-3,8-diazabicyclo[3.2.1]oct-8- |
| | yl)methanone |
| 20 | PCT/CN2017/088237, Ex. 11: 3-{[2-(4-chlorophenyl)imidazo[1,2-a]pyridin-3-yl]methyl}-8- |
| | oxa-3,10-diazabicyclo[4.3.1]dec-10-yl](3-fluoro-6-methoxypyridin-2-yl)methanone |
| | (enantiomer 1) |
| 21 | PCT/CN2017/088237, Ex. 12: 3-{[2-(4-chlorophenyl)imidazo[1,2-a]pyridin-3-yl]methyl}-8- |
| | oxa-3,10-diazabicyclo[4.3.1]dec-10-yl](3-fluoro-6-methoxypyridin-2-yl)methanone |
| | (enantiomer 2) |
| 22 | PCT/CN2017/088237, Ex. 13: 3-{[2-(4-chlorophenyl)imidazo[1,2-a]pyrimidin-3- |
| | yl]methyl}-8-oxa-3,10-diazabicyclo[4.3.1]dec-10-yl](3-fluoro-6-methoxypyridin-2- |
| | yl)methanone (enantiomer 1) |
| 23 | PCT/CN2017/088237, Ex. 14: 3-{[2-(4-chlorophenyl)imidazo[1,2-a]pyrimidin-3- |
| | yl]methyl}-8-oxa-3,10-diazabicyclo[4.3.1]dec-10-yl](3-fluoro-6-methoxypyridin-2- |
| | yl)methanone (enantiomer 2) |
| 24 | PCT/CN2017/088237, Ex. 15: [3-{[2-(5-chloropyridin-2-yl)imidazo[1,2-a]pyridin-3- |
| | yl]methyl}-3,9-diazabicyclo[4.2.1]non-9-yl](3-fluoro-6-methoxypyridin-2-yl)methanone |
| | (enantiomer 1) |
| 25 | PCT/CN2017/088237, Ex. 38: [6-{[2-(4-chlorophenyl)imidazo[1,2-a]pyridin-3-yl]methyl}- |
| | 2,6-diazabicyclo[3.2.2]non-2-yl](3-fluoro-6-methoxypyridin-2-yl)methanone (enantiomer 1) |
| 26 | PCT/CN2017/088237, Ex. 82: [3-{[2-(4-chlorophenyl)imidazo[1,2-a]pyrimidin-3- |
| | yl]methyl}-3,9-diazabicyclo[4.2.1]non-9-yl](3-fluoro-6-methoxypyridin-2-yl)methanone |
| | (enantiomer 1) |
| 27 | PCT/CN2017/088237, Ex. 101: (3-chloro-6-methoxypyridin-2-yl)[3-{[2-(4- |
| | isopropylphenyl)imidazo[1,2-a]pyrimidin-3-yl]methyl}-3,9-diazabicyclo[4.2.1]nonan-9- |
| | yl]methanone (enantiomer 1) |
| 28 | PCT/CN2017/088237, Ex. 102: (3-fluoro-6-methoxypyridin-2-yl)[3-{[2-(4- |
| | isopropylphenyl)imidazo[1,2-a]pyrimidin-3-yl]methyl}-3,9-diazabicyclo[4.2.1]nonan-9- |
| | yl]methanone (enantiomer 1) |
| 29 | PCT/CN2017/088237, Ex. 104: [3-{[2-(4-isopropylphenyl)imidazo[1,2-a]pyrimidin-3- |
| | yl]methyl}-3,9-diazabicyclo[4.2.1]nonan-9-yl](6-methoxypyridin-2-yl)methanone |
| | (enantiomer 1) |

and also a hydrate, solvate, polymorph or metabolite thereof or a pharmaceutically acceptable salt thereof.

Particular preference is given to compounds selected from the group comprising the compounds

| Example | Name |
|---|---|
| 6 | WO 2017/097792, Ex. 3: (4-{[2-(4-chlorophenyl)imidazo[1,2-a]pyridin-3- |
| | yl]methyl}piperazin-1-yl)(6-methoxypyridin-2-yl)methanone |
| 7 | WO 2017/097792, Ex. 4: (4-{[2-(4-bromophenyl)imidazo[1,2-a]pyridin-3- |
| | yl]methyl}piperazin-1-yl)(2-fluorophenyl)methanone |
| 10 | WO 2018/015196, Ex. 21: (−)-[(1S,4S)-5-{[2-(4-chlorophenyl)imidazo[1,2-a]pyridin-3- |
| | yl]methyl}-2,5-diazabicyclo[2.2.2]oct-2-yl](6-methoxypyridin-2-yl)methanone |
| | (enantiomer 1) |
| 11 | WO 2018/015196, Ex. 24: (5-{[2-(5-chloropyridin-2-yl)imidazo[1,2-a]pyridin-3-yl]methyl}- |
| | 2,5-diazabicyclo[2.2.2]oct-2-yl)-(3-fluoro-6-methoxypyridin-2-yl)methanone (enantiomer 1) |
| 12 | WO 2018/015196, Ex. 26: (−)-(5-{[2-(5-chloropyridin-2-yl)imidazo[1,2-a]pyridin-3- |
| | yl]methyl}-2,5-diazabicyclo[2.2.2]oct-2 yl)-(6-methoxypyridin-2-yl)methanone |
| | (enantiomer 1) |
| 14 | WO 2018/015196, Ex. 126: (3-fluoro-6-methoxypyridin-2-yl)(5-{[2-(6-isopropylpyridin-3- |
| | yl)imidazo[1,2-a]pyridin-3-yl]methyl}-2,5-diazabicyclo[2.2.2]oct-2-yl)methanone |
| | (enantiomer 1) |
| 17 | EP17176046.5, Ex. 26: (3-chloro-6-methoxypyridin-2-yl)(5-{[2-(4- |
| | isopropylphenyl)imidazo[1,2-a]pyrimidin-3-yl]methyl}-2,5-diazabicyclo[2.2.2]oct-2- |
| | yl)methanone (enantiomer 2) |
| 19 | EP17176046.5, Ex. 29: (3-chloro-6-methoxypyridin-2-yl)(3-{[2-(4- |
| | isopropylphenyl)imidazo[1,2-a]pyrimidin-3-yl]methyl}-3,8-diazabicyclo[3.2.1]oct-8- |
| | yl)methanone |
| 21 | PCT/CN2017/088237, Ex. 12: 3-{[2-(4-chlorophenyl)imidazo[1,2-a]pyridin-3-yl]methyl}-8- |
| | oxa-3,10-diazabicyclo[4.3.1]dec-10-yl](3-fluoro-6-methoxypyridin-2-yl)methanone |
| | (enantiomer 2) |
| 24 | PCT/CN2017/088237, Ex. 15: [3-{[2-(5-chloropyridin-2-yl)imidazo[1,2-a]pyridin-3- |
| | yl]methyl}-3,9-diazabicyclo[4.2.1]non-9-yl](3-fluoro-6-methoxypyridin-2-yl)methanone |
| | (enantiomer 1) |
| 25 | PCT/CN2017/088237, Ex. 38: [6-{[2-(4-chlorophenyl)imidazo[1,2-a]pyridin-3-yl]methyl}- |
| | 2,6-diazabicyclo[3.2.2]non-2-yl](3-fluoro-6-methoxypyridin-2-yl)methanone (enantiomer 1) |
| 26 | PCT/CN2017/088237, Ex. 82: [3-{[2-(4-chlorophenyl)imidazo [1,2-a]pyrimidin-3- |
| | yl]methyl}-3,9-diazabicyclo[4.2.1]non-9-yl](3-fluoro-6-methoxypyridin-2-yl)methanone |
| | (enantiomer 1) |

and also a hydrate, solvate, polymorph or metabolite thereof or a pharmaceutically acceptable salt thereof.

Very particular preference is given to compounds selected from the group comprising the compounds

| Example | Name |
|---|---|
| 6 | WO 2017/097792, Ex. 3: (4-{[2-(4-chlorophenyl)imidazo[1,2-a]pyridin-3- |
| | yl]methyl}piperazin-1-yl)(6-methoxypyridin-2-yl)methanone |
| 11 | WO 2018/015196, Ex. 24: (5-{[2-(5-chloropyridin-2-yl)imidazo[1,2-a]pyridin-3-yl]methyl}- |
| | 2,5-diazabicyclo[2.2.2]oct-2-yl)-(3-fluoro-6-methoxypyridin-2-yl)methanone (enantiomer 1) |
| 12 | WO 2018/015196, Ex. 26: (−)-(5-{[2-(5-chloropyridin-2-yl)imidazo[1,2-a]pyridin-3- |
| | yl]methyl}-2,5-diazabicyclo[2.2.2]oct-2 yl)-(6-methoxypyridin-2-yl)methanone |
| | (enantiomer 1) |
| 14 | WO 2018/015196, Ex. 126: (3-fluoro-6-methoxypyridin-2-yl)(5-{[2-(6-isopropylpyridin-3- |
| | yl)imidazo[1,2-a]pyridin-3-yl]methyl}-2,5-diazabicyclo[2.2.2]oct-2-yl)methanone |
| | (enantiomer 1) |
| 19 | EP17176046.5, Ex. 29: (3-chloro-6-methoxypyridin-2-yl)(3-{[2-(4- |
| | isopropylphenyl)imidazo[1,2-a]pyrimidin-3-yl]methyl}-3,8-diazabicyclo[3.2.1]oct-8- |
| | yl)methanone |
| 21 | PCT/CN2017/088237, Ex. 12: 3-{[2-(4-chlorophenyl)imidazo[1,2-a]pyridin-3-yl]methyl}-8- |
| | oxa-3,10-diazabicyclo[4.3.1]dec-10-yl](3-fluoro-6-methoxypyridin-2-yl)methanone |
| | (enantiomer 2) |
| 24 | PCT/CN2017/088237, Ex. 15: [3-{[2-(5-chloropyridin-2-yl)imidazo[1,2-a]pyridin-3- |
| | yl]methyl}-3,9-diazabicyclo[4.2.1]non-9-yl](3-fluoro-6-methoxypyridin-2-yl)methanone |
| | (enantiomer 1) |

and also a hydrate, solvate, polymorph or metabolite thereof or a pharmaceutically acceptable salt thereof.

Stable pharmaceutical formulations according to the invention are also those formulations in which the at least one inhibitor of the TASK-1 and/or TASK-3 channel is selected from the group consisting of

(4-{[2-(4-chlorophenyl)imidazo[1,2-a]pyridin-3-yl]methyl}piperazin-1-yl)(6-methoxypyridin-2-yl)methanone and/or (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone and also a hydrate, solvate, polymorph or metabolite thereof or a pharmaceutically acceptable salt thereof.

Stable pharmaceutical formulations according to the invention are also those formulations in which the at least one inhibitor of the TASK-1 and/or TASK-3 channel is (4-{[2-(4-chlorophenyl)imidazo[1,2-a]pyridin-3-yl]methyl}piperazin-1-yl)(6-methoxypyridin-2-yl)methanone.

Stable pharmaceutical formulations according to the invention are also those formulations in which the at least one inhibitor of the TASK-1 and/or TASK-3 channel is (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

A further embodiment of the present invention are the stable pharmaceutical formulations according to the invention for nasal or pharyngeal administration for the treatment and/or prevention of diseases.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apneas, central sleep apneas, snoring, cardiac arrhythmias, arrhythmias, neurodegenerative disorders, neuroinflammatory disorders and neuroimmunological disorders.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apneas, central sleep apneas, snoring, cardiac arrhythmias, arrhythmias, neurodegenerative disorders, neuroinflammatory disorders and neuroimmunological disorders, wherein the nasal or pharyngeal administration is aided by nasal sprays, nasal drops, nasal solutions, powder inhalers, nebulizers, metered dose aerosols or semisolid gels.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apneas, central sleep apneas, snoring, cardiac arrhythmias, arrhythmias, neurodegenerative disorders, neuroinflammatory disorders and neuroimmunological disorders.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apneas, central sleep apneas, snoring, cardiac arrhythmias, arrhythmias, neurodegenerative disorders, neuroinflammatory disorders and neuroimmunological disorders.

A further embodiment of the present invention are the stable pharmaceutical formulations according to the invention for nasal or pharyngeal administration for use in a method for the treatment and/or prevention of obstructive sleep apneas or snoring, comprising:

a therapeutically effective amount of the inhibitor of the TASK-1 and/or TASK-3 channel (4-{[2-(4-chlorophenyl)imidazo[1,2-a]pyridin-3-yl]methyl}piperazin-1-yl)(6-methoxypyridin-2-yl)methanone or (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone and/or a hydrate, solvate, polymorph or metabolite thereof or a pharmaceutically acceptable salt thereof in 1.0 to 21% by weight of polysorbate 80, 0.001% by weight to 0.2% by weight of butylated hydroxyanisole and at least one further auxiliary which are dissolved in phosphate or HEPES buffer solution at a substance concentration of 25 to 200 mM, with the pH of the formulation being adjusted to 6.8 to 8.2.

A further embodiment of the present invention are the stable pharmaceutical formulations according to the invention for nasal or pharyngeal administration for use in a method for the treatment and/or prevention of obstructive sleep apneas or snoring, comprising:

a therapeutically effective amount of the inhibitor of the TASK-1 and/or TASK-3 channel (4-{[2-(4-chlorophenyl)imidazo[1,2-a]pyridin-3-yl]methyl}piperazin-1-yl)(6-methoxypyridin-2-yl)methanone and/or a hydrate, solvate, polymorph or metabolite thereof or a pharmaceutically acceptable salt thereof in 1.0 to 21% by weight of polysorbate 80, 0.001% by weight to 0.2% by weight of butylated hydroxyanisole, 0.3 to 24.5% by weight of glycerol and at least one further auxiliary which are dissolved in phosphate or HEPES buffer solution at a substance concentration of 25 to 200 mM, with the pH of the formulation being adjusted to 6.8 to 8.2, preferably 6.8 to 7.8.

A further embodiment of the present invention are the stable pharmaceutical formulations according to the invention for nasal or pharyngeal administration for use in a method for the treatment and/or prevention of obstructive sleep apneas or snoring, comprising:

a therapeutically effective amount of the inhibitor of the TASK-1 and/or TASK-3 channel (4-{[2-(4-chlorophenyl)imidazo[1,2-a]pyridin-3-yl]methyl}piperazin-1-yl)(6-methoxypyridin-2-yl)methanone and/or a hydrate, solvate, polymorph or metabolite thereof or a pharmaceutically acceptable salt thereof in 1.0 to 21% by weight of polysorbate 80, 0.001% by weight to 0.2% by weight of butylated hydroxyanisole, 3 to 60% by weight of PEG 400 and at least one further auxiliary which are dissolved in phosphate or HEPES buffer solution at a substance concentration of 25 to 200 mM, with the pH of the formulation being adjusted to 6.8 to 8.2, preferably 6.8 to 7.8.

A further embodiment of the present invention are the stable pharmaceutical formulations according to the invention for nasal or pharyngeal administration for use in a method for the treatment and/or prevention of obstructive sleep apneas or snoring, comprising:

a therapeutically effective amount of the inhibitor of the TASK-1 and/or TASK-3 channel (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone and/or a hydrate, solvate, polymorph or metabolite thereof or a pharmaceutically acceptable salt thereof in 1.0 to 21% by weight of polysorbate 80, 0.001% by weight to 0.2% by weight of butylated hydroxyanisole, 0.05 to 0.25% by weight of sucralose and at least one further auxiliary which are dissolved in phosphate or HEPES buffer solution at a substance concentration of 25 to 200 mM, with the pH of the formulation being adjusted to 6.8 to 8.2, preferably 6.8 to 7.8.

The formulations of the invention can be used alone or, if required, in combination with one or more other pharmacologically active substances, provided that this combination does not lead to undesirable and unacceptable side effects. The present invention therefore further provides medicaments comprising at least one of the formulations of the invention and one or more further active ingredients, especially for treatment and/or prevention of the aforementioned disorders. Preferred examples of combination active ingredients suitable for this purpose include:
respiratory stimulants, by way of example and with preference theophylline, doxapram, nikethamide or caffeine;
psychostimulants, by way of example and with preference modafinil or armodafinil;
amphetamines and amphetamine derivatives, by way of example and with preference amphetamine, metamphetamine or methylphenidate;
serotonin reuptake inhibitors, by way of example and with preference fluoxetine, paroxetine, citalopram, escitalopram, sertraline, fluvoxamine or trazodone;
serotonin precursors, by way of example and with preference L-tryptophan;
selective serotonin noradrenaline reuptake inhibitors, by way of example and with preference venlafaxine or duloxetine;
noradrenergic and specific serotonergic antidepressants, by way of example and with preference mirtazapine;
selective noradrenaline reuptake inhibitors, by way of example and with preference reboxetine;
tricyclic antidepressants, by way of example and with preference amitriptyline, protriptyline, doxepine, trimipramine, imipramine, clomipramine or desipramine;
alpha2-adrenergic agonists, by way of example and with preference clonidine;
GABA agonists, by way of example and with preference baclofen;
alpha sympathomimetics, by way of example and with preference xylometazoline, oxymetazoline, phenylephrine, naphazoline, tetryzoline or tramazoline;
glucocorticoids, by way of example and with preference fluticasone, budesonide, beclometasone, mometasone, tixocortol or triamcinolone;
cannabinoid receptor agonists;
carboanhydrase inhibitors, by way of example and with preference acetazolamide, methazolamide or diclofenamide;
opioid and benzodiazepine receptor antagonists, by way of example and with preference flumazenil, naloxone or naltrexone;
cholinesterase inhibitors, by way of example and with preference neostigmine, pyridostigmine, physostigmine, donepezil, galantamine or rivastigmine;
N-methyl-D-aspartate and glutamate antagonists, by way of example and with preference amantadine, memantine or sabeluzole;
nicotine receptor agonists;
leukotriene receptor antagonists, by way of example and with preference montelukast or tipelukast;
dopamine receptor antagonists, by way of example and with preference dromperidone, metoclopramide or benzamide, butyrophenone or phenothiazine derivatives;
appetite suppressants, by way of example and with preference sibutramine, topiramate, phentermine, lipase inhibitors or cannabinoid receptor antagonists;
proton pump inhibitors, by way of example and with preference pantoprazole, omeprazole, esomeprazole, lansoprazole or rabeprazole;
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil, tadalafil, udenafil, dasantafil, avanafil, mirodenafil or lodenafil;
NO- and heme-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat, vericiguat and the compounds described in WO 00/06568, WO 00/06569, WO 02/42301, WO 03/095451, WO 2011/147809, WO 2012/004258, WO 2012/028647 and WO 2012/059549;
prostacyclin analogs and IP receptor agonists, by way of example and with preference iloprost, beraprost, treprostinil, epoprostenol or selexipag;
endothelin receptor antagonists, by way of example and with preference bosentan, darusentan, ambrisentan or sitaxsentan;
compounds which inhibit human neutrophile elastase (HNE), by way of example and with preference sivelestat or DX-890 (reltran);
compounds which inhibit the degradation and alteration of the extracellular matrix, by way of example and with preference inhibitors of the matrix metalloproteases (MMPs), especially inhibitors of stromelysin, collagenases, gelatinases and aggrecanases (in this context particularly of MMP-1, MMP-3, MMP-8, MMP-9, MMP-10, MMP-11 and MMP-13) and of metalloelastase (MMP-12);
compounds which block the binding of serotonin to its receptor, by way of example and with preference antagonists of the 5-HT₂B receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which influence the energy metabolism of the heart, by way of example and with preference etomoxir, dichloroacetate, ranolazine or trimetazidine;
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
anti-obstructive agents as used, for example, for treatment of chronic obstructive pulmonary disease (COPD) or bronchial asthma, by way of example and with preference from the group of the inhalatively or systemically administered agonists of the beta-adrenergic receptor (beta-mimetics) and the inhalatively administered anti-muscarinergic substances;
antiinflammatory, immunomodulating, immunosuppressive and/or cytotoxic agents, by way of example and with preference from the group of the systemically or inhalatively administered corticosteroids and also dimethyl fumarate, fingolimod, glatiramer acetate, β-interferons, natalizumab, teriflunomide, mitoxantrone, immunoglobulins, acetylcysteine, montelukast, tipelukast, azathioprine, cyclophosphamide, hydroxycarbamide, azithromycin, interferon-γ, pirfenidone or etanercept;
antifibrotic agents, by way of example and with preference lysophosphatidic acid receptor 1 (LPA-1) antagonists, CTGF inhibitors, IL-4 antagonists, IL-13 antagonists, TGF-β-antagonists or pirfenidone;
antithrombotic agents, by way of example and with preference from the group of platelet aggregation inhibitors, the anticoagulants and the profibrinolytic substances;
hypotensive active ingredients, by way of example and with preference from the group of the calcium antagonists, angiotensin AII antagonists, ACE inhibitors, vasopeptidase inhibitors, endothelin antagonists, renin inhibitors, alpha receptor blockers, beta receptor blockers, mineralocorticoid receptor antagonists and also the diuretics; and/or
active ingredients altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the formulations of the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the formulations of the invention are administered in combination with an antimuscarinergic substance, by way of example and with preference ipratropium bromide, tiotropium bromide or oxitropium bromide.

In a preferred embodiment of the invention, the formulations of the invention are administered in combination with a corticosteroid, by way of example and with preference prednisone, prednisolone, methylprednisolone, triamcinolone, dexamethasone, betamethasone, beclomethasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the formulations according to the invention are administered in combination with a platelet aggregation inhibitor, by way of example and with preference aspirin, clopidogrel, ticlopidine or dipyridamole.

In a preferred embodiment of the invention, the formulations of the invention are administered in combination with a thrombin inhibitor, by way of example and with preference ximelagatran, melagatran, dabigatran, bivalirudin or clexane.

In a preferred embodiment of the invention, the formulations of the invention are administered in combination with a GPIIb/IIIa antagonist, by way of example and with preference tirofiban or abciximab.

In a preferred embodiment of the invention, the formulations of the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the formulations according to the invention are administered in combination with heparin or with a low molecular weight (LMW) heparin derivative.

In a preferred embodiment of the invention, the formulations according to the invention are administered in combination with a vitamin K antagonist, by way of example and with preference coumarin.

Antihypertensives are preferably understood as meaning compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, and diuretics.

In a preferred embodiment of the invention, the formulations according to the invention are administered in combination with a calcium antagonist, by way of example and with preference nifedipine, amlodipine, verapamil or diltiazem.

In a preferred embodiment of the invention, the formulations according to the invention are administered in combination with an alpha-1 receptor blocker, by way of example and with preference prazosin.

In a preferred embodiment of the invention, the formulations according to the invention are administered in combination with a beta receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the formulations according to the invention are administered in combination with an angiotensin AII antagonist, by way of example and with preference losartan, candesartan, valsartan, telmisartan or embusartan.

In a preferred embodiment of the invention, the formulations according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the formulations according to the invention are administered in combination with an endothelin antagonist, by way of example and with preference bosentan, darusentan, ambrisentan or sitaxsentan.

In a preferred embodiment of the invention, the formulations according to the invention are administered in combination with a renin inhibitor, by way of example and with preference aliskiren, SPP-600 or SPP-800.

In a preferred embodiment of the invention, the formulations according to the invention are administered in combination with a mineralocorticoid receptor antagonist, by way of example and with preference spironolactone, eplerenone or finerenone.

In a preferred embodiment of the invention, the formulations according to the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerolin, isosorbide, mannitol, amiloride or triamterene.

Modifiers of lipid metabolism are preferably understood as meaning compounds from the group of CETP inhibitors, thyroid receptor agonists, cholesterol synthesis inhibitors such as HMG-CoA reductase inhibitors or squalene synthesis inhibitors, ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and lipoprotein(a) antagonists.

In a preferred embodiment of the invention, the formulations according to the invention are administered in combination with a CETP inhibitor, by way of example and with preference torcetrapib (CP-529 414), JJT-705 or CETP vaccine (Avant).

In a preferred embodiment of the invention, the formulations according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the formulations according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the formulations according to the invention are administered in combination with a squalene synthesis inhibitor, by way of example and with preference BMS-188494 or TAK-475.

In a preferred embodiment of the invention, the formulations according to the invention are administered in combination with an ACAT inhibitor, by way of example and with preference avasimibe, melinamide, pactimibe, eflucimibe or SMP-797.

In a preferred embodiment of the invention, the formulations according to the invention are administered in combination with an MTP inhibitor, by way of example and with preference implitapide, BMS-201038, R-103757 or JTT-130.

In a preferred embodiment of the invention, the formulations according to the invention are administered in combination with a PPAR-gamma agonist, by way of example and with preference pioglitazone or rosiglitazone.

In a preferred embodiment of the invention, the formulations according to the invention are administered in combination with a PPAR-delta agonist, by way of example and with preference GW 501516 or BAY 68-5042.

In a preferred embodiment of the invention, the formulations according to the invention are administered in combination with a cholesterol absorption inhibitor, by way of example and with preference ezetimibe, tiqueside or pamaqueside.

In a preferred embodiment of the invention, the formulations according to the invention are administered in combination with a lipase inhibitor, by way of example and with preference orlistat.

In a preferred embodiment of the invention, the formulations according to the invention are administered in combination with a polymeric bile acid adsorber, by way of example and with preference cholestyramine, colestipol, colesolvam, CholestaGel or colestimide.

In a preferred embodiment of the invention, the formulations according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the formulations according to the invention are administered in combination with a lipoprotein(a) antagonist, by way of example and with preference gemcabene calcium (CI-1027) or nicotinic acid.

Particular preference is given to combinations of the formulations according to the invention with one or more further active ingredients selected from the group consisting of respiratory stimulants, psychostimulants, serotonin reuptake inhibitors, noradrenergic, serotonergic and tricyclic antidepressants, sGC stimulators, mineralocorticoid receptor antagonists, antiinflammatory agents, immunomodulators, immunosuppressants and cytotoxic agents.

If required, the formulations according to the invention can also be employed in conjunction with the use of one or more medical technical devices or auxiliaries, provided that this does not lead to unwanted and unacceptable side effects. Medical devices and auxiliaries suitable for such a combined application are, by way of example and with preference:
devices for positive airway pressure ventilation, by way of example and with preference CPAP (continuous positive airway pressure) devices, BiPAP (bilevel positive airway pressure) devices and IPPV (intermittent positive pressure ventilation) devices;
neurostimulators of the Nervus hypoglossus;
intraoral auxiliaries, by way of example and with preference protrusion braces;
nasal disposable valves;
nasal stents.

In one embodiment, the dosage in the case of intranasal administration is about 0.1 μg to 500 μg per day. In a further embodiment, the dosage in the case of intranasal administration is about 1 μg to 250 μg per day. In a further embodiment, the dosage in the case of intranasal administration is about 1 μg to 100 μg per day. In a further embodiment, the dose of about 0.1 μg to 500 μg per day or of about 1 μg to 250 μg per day or of about 1 μg to 100 μg per day, is administered once daily by the intranasal route before sleeping. In one embodiment, the dose of about 0.1 μg to 500 μg per day or of about 1 μg to 250 μg per day or of about 1 μg to 100 μg per day, is administered once daily with half to each nostril. In one embodiment, the dose of about 0.1 μg to 500 μg per day or of about 1 μg to 250 μg per day or of about 1 μg to 100 μg per day, is administered once daily with half to each nostril before sleeping.

It may nevertheless be necessary in some cases to depart from the stated amounts, specifically as a function of body weight, route of administration, individual response to the active ingredient, nature of the formulation and time at which or interval over which administration takes place. Thus in some cases it may be sufficient to manage with less than the abovementioned minimum amount, whereas in other cases it is necessary to exceed the cited upper limit. If administering larger amounts, it may be advisable to divide them into several individual doses over the day.

### EXAMPLES

List of abbreviations

| | |
|---|---|
| OSA | Obstructive sleep apnea |
| PEG 400 | Polyethylene glycol 400, CAS No.: 25322-68-3 |
| TWEEN ® 80 | polysorbate 80; CAS No.: 9005-65-6 |
| ESTD | external standard method |
| PP | phosphate buffer (disodium hydrogen phosphate |
| | and sodium dihydrogen phosphate) |
| BHA | butylated hydroxyanisole |
| TASK | TWIK-related acid-sensitive K⁺ channel |
| HPLC | High Performance Liquid Chromatography |
| WFI | water for injection |
| ESTD | external standard |
| rpm | rounds per minute |
| h | hours |
| min | minutes |

### HPLC Methods

### Method 1 for (4-{[2-(4-chlorophenyl)imidazo[1,2-a]pyridin-3-yl]methyl}piperazin-1-yl)(6-methoxypyridin-2-yl)methanone

The undiluted samples are analyzed by reverse-phase HPLC on a Hewlett-Packard/Agilent HPLC and UHPLC instrument (DE). 2.0 μl of the sample solution were then applied to a metal column of stainless steel, e.g. Agilent Eclipse Plus RRHD C18 (150 mm×3.0 mm having a particle size of 1.8 μm) which is kept at a temperature of 25° C. (flow rate 0.5 ml/min).

The samples were analyzed using a B gradient of 10-45% (v/v) over 10 min, followed by 45-80% (v/v) over 5 min and then 5 min at 80% (v/v), with a mobile phase consisting of a solvent A (H₂O, with 1 ml of trifluoroacetic acid) and a solvent B (acetonitrile; Riedel-de Haën, DE with 1 ml of trifluoroacetic acid). The formulations are examined by the external standard method (ESTD) using a UV detector at 238 nm.

### Method 2 for (3-chloro-6-methoxypyridin-2-yl)(3-{[2-(4-isopropylphenyl)imidazo[1,2-a]pyrimidin-3-yl]methyl}-3.8-diazabicyclo[3.2.1]oct-8-yl)methanone

The samples are diluted with a mixture of water and methanol and then analyzed by reverse-phase HPLC on a Hewlett-Packard/Agilent HPLC or UHPLC instrument (DE). 3.0 μl of the sample solution were then applied to a metal column of stainless steel, e.g. Waters Acquity UPLC HSS T3 (50 mm×2.1 mm having a particle size of 1.8 μm) which is kept at a temperature of 40° C. (flow rate 10 ml/min).

The samples were analyzed using a B gradient of 5-30% (v/v) over 2.5 min, followed by 30-50% (v/v) over 5.5 min, followed by 50-80% (v/v) over 1 min and then 1 min at 80% (v/v), with a mobile phase consisting of a solvent A (0.77 g ammonium acetate/1 l of H₂O, adjusted to a pH of about 9 with ammonia) and a solvent B (acetonitrile; Riedel-de Haën, DE). The formulations are examined by the external standard method (ESTD) using a UV detector at 220 nm for content and area percent of degradation products.

### Method 3 for (3-chloro-6-methoxypyridin-2-yl)(3-{[2-(4-isopropylphenyl)imidazo[1,2-a]pyrimidin-3-yl]methyl}-3.8-diazabicyclo[3.2.1]oct-8-yl)methanone

The samples are diluted with a mixture of water and acetonitrile and then analyzed by reverse-phase HPLC on a Hewlett-Packard/Agilent HPLC or UHPLC instrument (DE). 4.0 μl of the sample solution were then applied to a metal column of stainless steel, e.g. Waters Acquity UPLC BEH Phenyl (100 mm×2.1 mm having a particle size of 1.7 μm) which is kept at a temperature of 50° C. (flow rate 0.5 ml/min).

The samples were analyzed using a B gradient of 5-51% (v/v) over 10 min, followed by 51-68% (v/v) over 7 min, followed by 68-90% (v/v) over 3 min and then 10 min at 90% (v/v), with a mobile phase consisting of a solvent A (114 mg ammonium acetate and 0.49 ml of glacial acetic acid/1 l of H₂O, pH about 4) and a solvent B (acetonitrile; Riedel-de Haën, DE). The formulations are examined by the external standard method (ESTD) using a UV detector at 238 nm.

### EXAMPLES OF THE ORDER OF PREPARATION

### Example 1 (Primary Solution A) is Added to B), According to the Invention)

A suitable vessel allowing stirring either with a magnetic stirrer bar or a blade agitator at a stirring speed of 200 to 300 rpm is chosen for the amphiphilic phase. 10% by volume (of the total material to be prepared) of polysorbate 80 are initially charged in this vessel. Subsequently, 0.02% (w/v) of butylated hydroxyanisole (BHA) is added with stirring and dissolved. By prior comminution of the BHA in a mortar, it is possible to reduce the dissolution time substantially. Only after the BHA is completely dissolved, 0.015% (w/v) of the micronized active compound (4-{[2-(4-chlorophenyl)imidazo[1,2-a]pyridin-3-yl]methyl}piperazin-1-yl)(6-methoxypyridin-2-yl)methanone is added with stirring.

The aqueous phase is prepared in a further vessel which is big enough for the entire material of the batch and also has to be fitted with a stirrer. About 70% of the WFI required is initially charged. With stirring at 200 to 300 rpm, the buffer salts disodium hydrogen phosphate and sodium dihydrogen phosphate dihydrate (total amount 0.063 mM phosphate, pH 7.0) are dissolved. Once the buffer salts are completely dissolved, 2.5% by volume of glycerol or 20% by volume of PEG 400 (see Table 2, 3, 4) are added and the mixture is stirred to homogeneity.

When the active compound is completely dissolved in the amphiphilic phase, the latter is added to the aqueous phase over a period of 30 minutes.

The vessel used for the amphiphilic phase is rinsed three times with WFI to ensure that the transfer of the amphiphilic phase is quantitative. Subsequently, the pH of the entire formulation is, if required, adjusted to between 6.8 and 7.2 using 10% strength HCl or 1N NaOH.

The entire formulation is then made up to the final mass using WFI.

### Example 2 (According to the Invention)

A suitable vessel allowing stirring either with a magnetic stirrer bar or a blade agitator at a stirring speed of 200 to 300 rpm and big enough for the entire material of the batch is chosen for the amphiphilic phase. 10% by volume (of the total material to be prepared) of polysorbate 80 are initially charged in this vessel. Subsequently, 0.02% (w/v) of BHA is added with stirring and dissolved. By prior comminution of the BHA in a mortar, it is possible to reduce the dissolution time substantially. Only after the BHA is completely dissolved, 0.015% (w/v) of the micronized active compound (4-{[2-(4-chlorophenyl)imidazo[1,2-a]pyridin-3-yl]methyl}piperazin-1-yl)(6-methoxypyridin-2-yl)methanone is added with stirring.

The aqueous phase is prepared in a further vessel which also has to be fitted with a stirrer. About 70% of the WFI required is initially charged. With stirring at 200 to 300 rpm, the buffer salts disodium hydrogen phosphate and sodium dihydrogen phosphate dihydrate (total amount 0.063 mM phosphate, pH 7.0) are dissolved. Once the buffer salts are completely dissolved, 2.5% by volume of glycerol or 20% by volume of PEG 400 (see Table 2) are added and the mixture is stirred to homogeneity.

When the active compound is completely dissolved in the amphiphilic phase, the entire aqueous phase is added to the amphiphilic phase over a period of 30 minutes.

If required, the pH is adjusted to between 6.8 and 7 using 10% strength HCl or 1N NaOH.

The entire formulation is then made up to the final mass using WFI.

### Example 3 (Comparative Example)

About 70% of the required WFI is initially charged in a suitable vessel which is big enough for the entire material of the batch and allows stirring either with a magnetic stirrer bar or a blade agitator at a stirring speed of 200 to 300 rpm. With stirring, the buffer salts disodium hydrogen phosphate and sodium dihydrogen phosphate dihydrate (total amount 0.063 mM phosphate, pH 7.0) are introduced into this vessel. Once the buffer salts are completely dissolved, 2.5% by volume of glycerol or 20% by volume of PEG 400 (see Table 2, 3, 4) are added and the mixture is stirred to homogeneity. Subsequently, over a period of 30 minutes, 10% by volume of polysorbate 80 are added with stirring. Once the polysorbate is completely dissolved, 0.02% (w/v) of BHA is added and dissolved. By prior comminution of the BHA in a mortar, it is possible to reduce the dissolution time substantially. Only after the BHA is completely dissolved, 0.015% (w/v) of the micronized active compound (4-{[2-(4-chlorophenyl)imidazo[1,2-a]pyridin-3-yl]methyl}piperazin-1-yl)(6-methoxypyridin-2-yl)methanone is added with stirring. Once the active compound is also completely dissolved, the pH of the entire formulation is, if required, adjusted to between 6.8 and 7.2 using 10% strength HCl or 1N NaOH.

The entire formulation is then made up to the final mass using WFI.

### Example 4 (According to the Invention)

20% by volume of PEG 400 (see Table 2) is initially charged in a suitable vessel which is big enough for the entire material of the batch and allows stirring either with a magnetic stirrer bar or a blade agitator at a stirring speed of 200 to 300 rpm. To this vessel, 0.02% (w/v) of BHA is added with stirring and dissolved. By prior comminution of the BHA in a mortar, it is possible to reduce the dissolution time substantially. Subsequently, 0.015% (w/v) of the micronized active compound (4-{[2-(4-chlorophenyl)imidazo[1,2-a]pyridin-3-yl]methyl}piperazin-1-yl)(6-methoxypyridin-2-yl)methanone is added with stirring. Once the active compound is also completely dissolved, about 70% of the required WFI is added and the mixture is stirred to homogeneity. Subsequently, the buffer salts disodium hydrogen phosphate and sodium dihydrogen phosphate dihydrate (total amount 0.063 mM phosphate, pH 7.0) are dissolved. Over a period of 30 minutes, 10% by volume of polysorbate 80 are then added with stirring. Once the polysorbate is also completely dissolved, the pH of the entire formulation is, if required, adjusted to between 6.8 and 7.2 using 10% strength HCl or 1N NaOH.

The entire formulation is then made up to the final mass using WFI.

### Example 5 (According to the Invention)

10% by volume of polysorbate 80 is initially charged in a suitable vessel which is big enough for the entire material of the batch and allows stirring either with a magnetic stirrer bar or a blade agitator at a stirring speed of 200 to 300 rpm. To this vessel, 0.02% (w/v) of BHA is added with stirring and dissolved. By prior comminution of the BHA in a mortar, it is possible to reduce the dissolution time substantially. Subsequently, 0.015% (w/v) of the micronized active compound (4-{[2-(4-chlorophenyl)imidazo[1,2-a]pyridin-3-yl]methyl}piperazin-1-yl)(6-methoxypyridin-2-yl)methanone is added with stirring. Once the active compound is also completely dissolved, 2.5% by volume of glycerol or 20% by volume of PEG 400 (see Table 2) are added and the mixture is stirred to homogeneity. Subsequently, about 70% of the WFI required is added. Once a homogeneous solution has formed, the buffer salts disodium hydrogen phosphate and sodium dihydrogen phosphate dihydrate (total amount 0.063 mM phosphate, pH 7.0) are added with stirring. Once the buffer salts are also dissolved, the pH of the entire formulation is, if required, adjusted to between 6.8 and 7.2 using 10% strength HCl or 1N NaOH.

The entire formulation is then made up to the final mass using WFI.

### Example 6 (According to the Invention)

10% by volume of polysorbate 80 is initially charged in a suitable vessel which is big enough for the entire material of the batch and allows stirring either with a magnetic stirrer bar or a blade agitator at a stirring speed of 200 to 300 rpm. To this vessel, 0.02% (w/v) of BHA is added with stirring and dissolved. By prior comminution of the BHA in a mortar, it is possible to reduce the dissolution time substantially. 0.015% (w/v) of the micronized active compound is then added with stirring. Once the active compound is also completely dissolved, about 70% of the required WFI is added and the mixture is stirred to homogeneity. Subsequently, the buffer salts disodium hydrogen phosphate and sodium dihydrogen phosphate dihydrate (total amount 0.063 mM phosphate, pH 7.0) are added and dissolved. 2.5% by volume of glycerol or 20% by volume of PEG 400 (see Table 2, 3, 4) are then added and the mixture again is stirred to homogeneity. Once the buffer salts have also gone into solution, the pH of the entire formulation is checked. If the pH is not between 6.8 and 7.2, it is readjusted using 10% strength HCl or 1N NaOH.

The entire formulation is then made up to the final mass using WFI.

### Example 7 (Comparative Example)

10% by volume of polysorbate 80 is initially charged in a suitable vessel which is big enough for the entire material of the batch and allows stirring either with a magnetic stirrer bar or a blade agitator at a stirring speed of 200 to 300 rpm. To this vessel, 0.015% (w/v) of the micronized active compound is added with stirring. Once the active compound is completely dissolved, about 70% of the required WFI is added. Once a homogeneous solution has formed, the buffer salts disodium hydrogen phosphate and sodium dihydrogen phosphate dihydrate (total amount 0.063 mM phosphate, pH 7.0) are added with stirring. 2.5% by volume of glycerol or 20% by volume of PEG 400 (see Table 2, 3, 4) are then added and the mixture again is stirred to homogeneity. Subsequently, 0.02% (w/v) of BHA is added with stirring and dissolved. By prior comminution of the BHA in a mortar, it is possible to reduce the dissolution time substantially. Once the BHA has also gone into solution, the pH of the entire formulation is checked. If the pH is not between 6.8 and 7.2, it is readjusted using 10% strength HCl or 1N NaOH.

The entire formulation is then made up to the final mass using WFI.

TABLE 2
Active compound employed: (4-{[2-(4-chlorophenyl)imidazo[1,2-a]
pyridin-3-yl]methyl}piperazin-1-yl)(6-methoxypyridin-2-yl)methanone

| | | Duration of the | | Degradation |
|---|---|---|---|---|
| Preparation | | preparation | Content | products |
| example | Formulation | [min] | [%] | (total) [%] |
| Example 1 | glycerol | 140 min | 98.15 | <0.10 |
| | PEG 400 | 100 min | 95.94 | <0.10 |
| Example 2 | glycerol | 150 min | 97.89 | <0.10 |
| | PEG 400 | 115 min | 97.00 | 0.41 |
| Example 3* | glycerol | >480 min | 98.45 | 0.59 |
| | PEG 400 | >440 min | 93.94 | 0.20 |
| Example 4 | glycerol | not possible | | |
| | PEG 400 | 140 min | 97.21 | <0.10 |
| Example 5 | glycerol | 110 min | 98.567 | <0.10 |
| | PEG 400 | 250 min | 96.09 | 0.95 |
| Example 6 | glycerol | 120 min | 97.32 | <0.10 |
| | PEG 400 | 195 min | 96.00 | <0.10 |
| Example 7* | glycerol | >360 min | 96.72 | 0.16 |
| | PEG 400 | >360 min | 96.75 | 0.19 |

*Comparative examples

For further embodiments of the process according to the invention, preparation examples 1, 3, 6 and 7 were carried out to evaluate the preparation time. The experiments were carried out for two processes of the invention according to Examples 1 and 6 and according to Comparative examples 3 and 7. In each case, sucralose was added after the addition of the buffer salts.

TABLE 3
Active compound employed: (4-{[2-(4-chlorophenyl)imidazo[1,2-a]
pyridin-3-yl]methyl}piperazin-1-yl)(6-methoxypyridin-2-yl)methanone

| | | Duration of the |
|---|---|---|
| Preparation | Formulation according to preparation | preparation |
| example | example with . . . | [min] |
| Example 1 | glycerol and additionally sucralose | 150 |
| | glycerol and HEPES (instead of PP) | 125 |
| Example 3* | glycerol and additionally sucralose | >480 |
| | glycerol and HEPES (instead of PP) | >480 |
| Example 6 | glycerol and additionally sucralose | 165 |
| | glycerol and HEPES (instead of PP) | 140 |
| Example 7* | glycerol and additionally sucralose | >360 |
| | glycerol and HEPES (instead of PP) | >360 |

TABLE 4
Active compound employed: (3-chloro-6-methoxypyridin-2-yl)(3-{[2-
(4-isopropylphenyl)imidazo[1,2-a]pyrimidin-3-yl]methyl}-3,8-
diazabicyclo[3.2.1]oct-8-yl)methanone

| | Formulation | Duration of |
|---|---|---|
| Preparation | according to preparation example | the preparation |
| example | without glycerol or PEG, but with . . . | [min] |
| Example 1 | additionally sucralose | 155 |
| | HEPES (instead of PP) and additionally | 160 |
| | sucralose | |
| Example 3* | additionally sucralose | >480 |
| | HEPES (instead of PP) and additionally | >600 |
| | sucralose | |
| Example 6 | additionally sucralose | 145 |
| | HEPES (instead of PP) and additionally | 180 |
| | sucralose | |
| Example 7* | additionally sucralose | >360 |
| | HEPES (instead of PP) and additionally | >360 |
| | sucralose | |

1: A process for producing stable pharmaceutical formulations, comprising a first step wherein at least one polyoxyethylenesorbitan fatty ester is initially charged as solubilizer and/or PEG 400 is initially charged as cosolvent, at least one antioxidant and a therapeutically effective amount of at least one inhibitor of the TASK-1 and/or TASK-3 channel or a hydrate, solvate, polymorph or metabolite thereof or a pharmaceutically acceptable salt are dissolved therein and subsequently at least one pH regulator, water and optionally glycerol, polyoxyethylene sorbitan fatty ester or PEG400 and optionally at least one sweetener are added, the pH of the resulting solution being between 6.8 and 8.2.

2: The process of claim 1, wherein at least one polyoxyethylene sorbitan fatty ester as solubilizer and/or PEG 400 as cosolvent is initially charged, the antioxidant is then added and subsequently a therapeutically effective amount of at least one inhibitor of the TASK-1 and/or TASK-3 channel or a hydrate, solvate, polymorph or metabolite thereof or a pharmaceutically acceptable salt is dissolved therein.

3: The process of claim 1, wherein initially
a primary solution (A) comprising at least one polyoxyethylene sorbitan fatty ester (polysorbate) and/or PEG 400 and also at least one antioxidant is prepared and, in a further step, a therapeutically effective amount of at least one inhibitor of the TASK-1 and/or TASK-3 channel or a hydrate, solvate, polymorph or metabolite thereof or a pharmaceutically acceptable salt is dissolved in this mixture, and this is added to a
solution (B) comprising at least one pH regulator, water and optionally glycerol or PEG400 and optionally at least one sweetener, the pH of the resulting solution being between 6.8 and 8.2.

4: The process of claim 1, wherein in a first step at least one polyoxyethylene sorbitan fatty ester is initially charged, the antioxidant is then added and subsequently a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone is dissolved therein and then at least one pH regulator, at least one sweetener and water are added.

5: The process of claim 1, wherein in a first step at least one polyoxyethylene sorbitan fatty ester is initially charged, the antioxidant is then added and subsequently a therapeutically effective amount of (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone is dissolved therein and then at least one pH regulator and water are added.

6: The process of claim 1, wherein in a first step at least one polyoxyethylene sorbitan fatty ester is initially charged, the antioxidant is then added and subsequently a therapeutically effective amount of (4-{[2-(4-chlorophenyl)imidazo[1,2-a]pyridin-3-yl]methyl}piperazin-1-yl)(6-methoxypyridin-2-yl)methanone is dissolved and then at least one pH regulator, glycerol or PEG 400, optionally a sweetener and water are added.

7: The process of claim 1, wherein in a first step PEG 400 is initially charged, the antioxidant is then added and subsequently a therapeutically effective amount of (4-{[2-(4-chlorophenyl)imidazo[1,2-a]pyridin-3-yl]methyl}piperazin-1-yl)(6-methoxypyridin-2-yl)methanone is dissolved and then at least one pH regulator, at least one polyoxyethylene sorbitan fatty ester, optionally a sweetener and water are added.

8: A pharmaceutical formulation, obtainable according to the process of claim 1.

9: The pharmaceutical formulation of claim 8, comprising:
1 to 210% by weight of a polyoxyethylene sorbitan fatty ester,
0.001 to 0.20% by weight of an antioxidant,
0.002 to 0.10% by weight of a therapeutically effective amount of at least one inhibitor of the TASK-1 and/or TASK-3 channel or a hydrate, solvate, polymorph or metabolite thereof or a pharmaceutically acceptable salt,
0.3 to 25% by weight of glycerol and
53.5 to 98% by weight of a buffer solution having a substance concentration of 25 to 200 mM.

10: The pharmaceutical formulation of claim 8, comprising:
1 to 20% by weight of a polyoxyethylene sorbitan fatty ester,
0.001 to 0.2% by weight of an antioxidant,
0.002 to 0.10% by weight of a therapeutically effective amount of at least one inhibitor of the TASK-1 and/or TASK-3 channel or a hydrate, solvate, polymorph or metabolite thereof or a pharmaceutically acceptable salt,
3.0 to 60% by weight of PEG 400 and
19 to 95.5% by weight of a buffer solution having a substance concentration of 25 to 200 mM.

11: The pharmaceutical formulation of claim 9, comprising 4-{[2-(4-chlorophenyl)imidazo[1,2-a]pyridin-3-yl]methyl}piperazin-1-yl)(6-methoxypyridin-2-yl)methanone.

12: The pharmaceutical formulation of claim 8, comprising:
1 to 21% by weight of a polyoxyethylene sorbitan fatty ester,
0.001 to 0.2% by weight of an antioxidant,
0.002 to 0.1% by weight of a therapeutically effective amount of at least one inhibitor of the TASK-1 and/or TASK-3 channel or a hydrate, solvate, polymorph or metabolite thereof or a pharmaceutically acceptable salt,
0.01 to 6% by weight of a sweetener and
72 to 98.5% by weight of a buffer solution having a substance concentration of 25 to 200 mM.

13: The pharmaceutical formulation of claim 8, comprising:
1.4 to 22.7% by weight of a polyoxyethylene sorbitan fatty ester,
0.001 to 0.2% by weight of an antioxidant,
0.002 to 0.1% by weight of a therapeutically effective amount of at least one inhibitor of the TASK-1 and/or TASK-3 channel or a hydrate, solvate, polymorph or metabolite thereof or a pharmaceutically acceptable salt,
0 to 4% by weight of a sweetener and
73 to 98.5% by weight of a buffer solution having a substance concentration of 25 to 200 mM.

14: The pharmaceutical formulation of claim 12, comprising (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.

15: A method for treatment or prevention of a disease, comprising administering to a human in need thereof a pharmaceutical formulation according to claim 8, wherein the pharmaceutical formulation is administered by nasal or pharyngeal administration.

16: A method for the treatment or prevention of respiratory disorders, sleep-related respiratory disorders, obstructive sleep apneas, central sleep apneas, snoring, cardiac arrhythmias, arrhythmias, neurodegenerative disorders, neuroinflammatory disorders and neuroimmunological disorders, comprising administering to a human in need thereof a pharmaceutical formulation according to claim 8, wherein the pharmaceutical formulation is administered by nasal or pharyngeal administration.

17: A method for the treatment or prevention of respiratory disorders, sleep-related respiratory disorders, obstructive sleep apneas, central sleep apneas, snoring, cardiac arrhythmias, arrhythmias, neurodegenerative disorders, neuroinflammatory disorders and neuroimmunological disorders, comprising administering to a human in need thereof a pharmaceutical formulation according to claim 8, wherein the pharmaceutical formulation is administered by nasal or pharyngeal administration, and wherein the nasal or pharyngeal administration is aided by nasal sprays, nasal drops, nasal solutions, powder inhalers, nebulizers, metered dose aerosols or semisolid gels.

18: The pharmaceutical formulation of claim 10, comprising 4-{[2-(4-chlorophenyl)imidazo[1,2-a]pyridin-3-yl]methyl}piperazin-1-yl)(6-methoxypyridin-2-yl)methanone.

19: The pharmaceutical formulation of claim 13, comprising (3-chloro-6-methoxypyridin-2-yl)(3-{[2-(4-isopropylphenyl)imidazo[1,2-a]pyrimidin-3-yl]methyl}-3,8-diazabicyclo[3.2.1]oct-8-yl)methanone.
